(19) United States
(12) Patent Application Publication
(10) Pub. No.: US 2003/0211133 A1
(43) Pub. Date: Nov. 13, 2003

(54) Ingestible composition for enhancing athletic performance

(75) Inventor: Kevin J. Meehan, Victor, ID (US)

Correspondence Address: Dale F. Regelman, Law Office of Dale F. Regelman, P.C., 4231 S. Fremont Avenue, Tucson, AZ, 85714, US

(73) Assignee: Special OPS Nutrition, L.L.C.

(21) Appl. No.: 10/386,786

(22) Filed: Mar. 12, 2003

Related U.S. Application Data
(60) Provisional application No. 60/379,638, filed on May 10, 2002.

(51) Int. Cl. A61K 38/46; A61K 31/56; A61K 35/78

(52) U.S. Cl. 424/439; 424/725; 514/23; 424/646; 424/654; 514/651; 514/393; 514/355; 514/52; 514/564; 514/171; 424/94.6

(57) ABSTRACT

An ingestible composition for enhancing athletic performance, which includes Methylcobalamine, a plurality of B vitamins, one or more Group VI element-containing compounds, one or more Group VIII element-containing compounds, and optionally one or more Group I element-containing compounds and one or more Group II element-containing compounds, Camitin, Citrulline, Thiamine, Lipoic Acid, Lysine, one or more proteolytic enzymes, one or more amylolytic enzymes, one or more lipolytic enzymes, one or more coenzymes, and one or more herbal extracts.

### FIELD OF THE INVENTION

This invention relates to an ingestible composition for enhancing athletic performance. In certain embodiments, Applicant's invention further relates to a water-based composition which is isotonic with normal bodily fluids.

### BACKGROUND OF THE INVENTION

There are a number of liquid compositions or diluted mixtures sold in commerce having names such as “Activity drinks,” “Sports drinks,” “Energy drinks,” “Nutrient drinks,” and the like. These beverages are advertised to ameliorate physiologic symptoms resulting from the loss of carbohydrates, electrolytes, vitamins, minerals, amino acids, and other important nutrients, during heavy exercise. As those skilled in the art will appreciate, athletic performance, i.e. physical exercise, comprises many different categories of activities, including activities requiring strength, speed, and/or endurance. As those skilled in the art will further appreciate, environmental factors, including temperature, air purity, elevation, humidity, and the like, can markedly affect a person's physical work capacity.

It is thought that muscle activity is primarily based on a very fundamental biochemical mechanism, the breakdown of energy-rich phosphate bonds. Adenosine triphosphate (“ATP”) is one source of such phosphate bonds at the cellular level. ATP is the direct source of energy for muscle work and, some believe, comprises the only form of chemical energy which can be converted by muscle tissue into mechanical work.

During high physical activity of the body the ATP level in the muscles diminishes rapidly. Several substrates are available as sources for replenishing the ATP. When there is low physical activity metabolism of fats is primarily responsible for ATP production. At higher activity rates, glycogen in the muscle is the major energy supply. The energy from glycogen is released in exercising muscles up to three times as fast as the energy from fat. It is known in the art that exercise of a moderate intensity cannot be maintained without sufficient carbohydrate stores within the body. Carbohydrates are the fuel from which body cells obtain energy for cellular activities and the major portion of carbohydrates utilized by the body are used for ATP production. The energy required for developing athletic activity, and indeed for all muscular work, comes primarily from the oxidation of glycogen stored in the muscles.

Glycogen can be used either relatively slowly via the complete glycolysis and oxidative phosphorylation to form carbon dioxide, water and 38 moles of ATP per mole of glucose. The basic biochemical pathway being: C₆H₁₂O₆+6O₂→6CO₂+6H₂O+energy (heat, ATP). This happens not all at once but in many small steps, to control the release of energy. Each step uses one or more enzymes; some use ATP for activation energy. The first process is sometimes called Glycolysis, where, using enzymes, glucose is cleaved into two pieces, and some ATP and NADH are formed. Subsequently, the Krebs cycle transfers electrons, H⁺ and energy from C—H bonds to NAD⁺, making NADH. In addition, some ATP is formed. The Krebs cycle occurs in the center of the mitochondrion (inside inner membrane).

Thereafter, an electron transport chain transfers the energy from NADH to produce more than 30 moles of ATP. This happens on the inner membrane of the mitochondrion. Energy is used in small steps to push H⁺ ions across the membrane. They pile up, then flow through an opening in ATP synthase (an enzyme), where the energy of the flow is used to make ATP.

When exercise is very intensive, i.e. so intensive that the respiratory and cardiovascular systems of the body do not have sufficient time to deliver oxygen to the muscles, the energy for this activity will be delivered almost exclusively from anaerobic metabolism, and much less ATP per molecule of glucose is produced.

Fatigue during high intensity exercise may be viewed as the result of a simple mismatching between the rate at which ATP is utilized and the rate at which ATP is produced in working muscles. The attention, given over the last two decades to the study of the limitations of ATP production, leads to the conclusion that the cause of fatigue may be the inability of the metabolic machinery to provide ATP fast enough for the energy needs of the working muscles to sustain force production.

Furthermore, during relatively extended periods of heavy muscle work, the work capacity of an individual is limited by several factors, such as low blood sugar concentration and loss of liquid by transpiration. In the last decade the use of liquid drinks containing carbohydrates during exercise has become more and more accepted as a stimulus during endurance performance. As a result, the prior art focuses exclusively on ingesting substantial amounts of carbohydrate in a liquid form during endurance competition events. The prior art further teaches that supplementation with carbohydrate containing fluids is useful to prolong exercise and improve the performance of high intensity endurance exercise. Benefits to be obtained are:

maintenance of fluid balance and an increase in the availability of carbohydrate, the primary substrate for the muscular ATP production.

Applicants have found, however, that it is not always logistically possible to consume large amounts of carbohydrate-containing beverages over extended periods of time. For example, heavy exercise in remote areas wherein any such beverages must first be carried for long distances prior to consumption renders such a prior art approach non-feasible. Moreover, although considerable amounts of carbohydrates can be ingested, not all of the exogenous carbohydrates emptied from the stomach are oxidize during exercise.

In addition, gastric emptying rate decreases with increasing carbohydrate concentration and osmolality. Consequently highly concentrated carbohydrate solutions have been observed to increase the frequency of gastrointestinal distress in endurance athletes. Certain timing issues can further complicate the consumption of large amounts of carbohydrates. The efficiency of ingested glucose in enhancing physical performance is dependent on the time at which the beverage is ingested before exercise. It is known in the art that glucose containing beverages produce an increase in plasma glucose peaking approximately 45 minutes after ingestion. Such an increase in plasma glucose, however, results in an increase in plasma insulin and a subsequent drop in plasma glucose during the initial period of the activity, resulting in quick exhaustion. Thus, ingestion of large amounts of carbohydrates prior to embarking on a lengthy period of vigorous physical activity in the afore-mentioned remote area scenario can be deleterious rather than advantageous.

Applicant's ingestible composition, and method using that composition, focus on facilitating at the cellular level both carbohydrate and non-carbohydrate metabolism. To the extent energy production can be shifted, partially at least, away from glycogen metabolism, the body's available glycogen resources are utilized at a reduced rate, thereby extending the body's ability to perform maximally at a high level of physical activity. Applicant's composition includes certain digestive enzymes that assist with pancreatic involvement as well as facilitating gastric function. Applicant's composition and method facilitate metabolism of fats by allowing, for example, long chain fatty acids to enter the mitochondrial matrix. By maximizing non-carbohydrate metabolic processes, Applicant's composition and method reserve glycogen resources stored in the muscle and liver. Applicant's composition and method further buffer metabolic acids at a high rate to maintain a desired pH at the cellular level.

### SUMMARY OF THE INVENTION

Applicant's invention includes an ingestible composition for enhancing athletic performance. Applicant's composition includes Methylcobalamine, a plurality of B vitamins, Camitin, Citrulline, Thiamin, Lipoic Acid, Lysine, Thiamine, one or more Group VI elements;

and one or more Group VIII elements. In certain embodiments, Applicant's composition further includes one or more proteolytic enzymes, one or more amylolytic enzymes, one or more lipolytic enzymes, one or more coenzyme, and one or more herbal extracts.

### DETAILED DESCRIPTION OF THE PREFERRED EMBODIMENTS

Applicant's composition optionally includes a plurality of vitamins, a plurality of digestive enzymes, a plurality of coenzymes, one or more herbs, one or more minerals, one or more electrolytes, and one or more derivatives of certain human hormones. As those skilled in the art will appreciate, a coenzyme is a substance that facilitates the proper functioning of an enzyme. Many such coenzymes comprise proteins that speed up the rate at which chemical reactions, including cellular metabolism of fats and carbohydrates, take place in the body.

Over the last decade or so, researchers have strongly implicated the toxic amino acid homocysteine in a variety of disease states. Homocysteine tends to accumulate in the body whenever Vitamin B₁₂ gets deficient. Folic acid deficiency can also lead to increased homocysteine levels—that's because folate and B₁₂, in their active “coenzyme” forms, are both necessary cofactors for the enzymatic conversion of homocysteine to methionine. Until recently it's been thought that the availability of folate was the most important determinant of the body's ability to remethylate homocysteine.

New research has revealed that vitamin B₁₂ is more important for homocysteine disposal than once believed. The coenzyme form of vitamin B₁₂ is known as Methylcobalamine. It's the only form of vitamin B₁₂ which can directly participate in homocysteine metabolism. In addition, converting homocysteine to methionine via Methylcobalamine generates an increased supply of SAMe (S-adenosyl methionine), the body's most important methyl donor. Indeed, some of the benefits of Methylcobalamine, such as protection from neurotoxicity, appear to derive from increased production of SAMe. Methylcobalamine has also been reported to be neurotrophic, or growth-promoting, for nerve cells, a property which may help regenerate central and peripheral nervous tissues damaged in disorders such as amyotrophic lateral sclerosis and diabetic peripheral neuropathy. Methylcobalamine is the most highly reduced form of vitamin B₁₂ possible; this makes Methylcobalamine a very potent reducing agent, i.e. antioxidant.

Applicant's composition includes Methylcobalamine. Using Applicant's composition and method, Methylcobalamine is provided at between about 0.6 milligram and about 5 milligrams per administration, i.e. upon ingestion of one dosage of Applicant's composition. In certain embodiments, Applicant's composition includes about 2.4 milligrams of Methylcobalamine.

Biotin, compound I, comprises a water soluble member of the vitamin B family. Biotin is sometimes called Vitamin H. In certain embodiments, Applicant's composition includes a racemic mixture of biotin stereoisomers. In certain embodiments, Applicant's composition includes D-biotin, shown below as Compound I. In certain embodiments, Applicant's composition includes a salt of racemic biotin or D-biotin. In certain embodiments, Applicant's composition includes biotin hydrochloride.

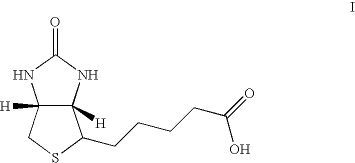

Using Applicant's composition and method, biotin is provided at between about 480 micrograms and about 3,000 micrograms per administration, i.e. upon ingestion of one dosage of Applicant's composition. In certain embodiments, Applicant's composition includes about 1920 micrograms of Methylcobalamine.

Pantathine, sometimes called Vitamin B₅ or Pantothenic Acid, another of the B complex vitamins, comprises a yellow viscous oil. Pantothenic acid is sometimes used as a calcium or sodium salt, calcium pantothenate for example. Pantothenic acid is present in all living cells and is very important to metabolism where it functions as part of the molecule called coenzyme A or CoA. As the coenzyme, pantothenic acid is important in cellular metabolism of carbohydrates and fats to release energy.

In certain embodiments, Applicant's composition includes Pantothenic acid. In certain embodiments, Applicant's composition includes calcium pantothenate. In certain embodiments, Applicant's composition includes sodium pantothenate. In certain embodiments, Applicant's composition includes potassium pantothenate. In certain embodiments, Applicant's composition includes magnesium pantothenate. Using Applicant's composition and method, Pantothenic acid and/or a pantothenate salt, is provided at between about 30 milligrams and about 250 milligrams per administration, i.e. upon ingestion of one dosage of Applicant's composition. In certain embodiments, Applicant's composition includes about 120 milligrams of Pantothenic acid.

Niacinamide, Inosital Hexaniacinate, is the amide form (more water soluble) of Niacin, sometimes called Vitamin B₃. Niacinamide assists cells to metabolize protein, carbohydrates, and fats. Applicant's composition includes Niacinamide. Using Applicant's composition and method, Niacinamide is provided at between about 90 milligrams and about 500 milligrams per administration, i.e. upon ingestion of one dosage of Applicant's composition. In certain embodiments, Applicant's composition includes about 360 milligrams of Niacinamide.

Carnitine plays a vital role in the transport of fatty acids across the inner mitochondrial membrane where these fatty acids are metabolized thereby producing a major source of muscular energy. L-Carnitine, compound II wherein R4, R5, and R6 are methyl, comprises a natural substance that helps release stored body fat (tryglycerides) into the bloodstream for use in cellular energy production.

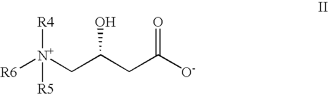

In addition, Carnitine boosts energy by stimulating the body's burning of fats (tryglycerides) as fuel. By burning fats as fuel, the body's supply of glycogen stored in the liver is spared for heavier exertion. Generally, the body will burn fat up to 75 to 80 percent of maximum exertion. As those skilled in the art will appreciate, metabolizing fat for energy spares the use of glycogen.

Inclusion of carnitine in Applicant's composition enhances lipid oxidation and thereby improves endurance performance by sparing endogenous carbohydrate. With respect to anaerobic activity, inclusion of carnitine in Applicant's composition improves performance by inhibiting lactic acid production. Using Applicant's composition and method, Carnitine is provided at between about 79.5 milligrams and about 500 milligrams per administration, i.e. upon ingestion of one dosage of Applicant's composition. In certain embodiments, Applicant's composition includes about 318 milligrams of L-Carnitine.

Betaine (trimethylglycine) functions very closely with choline, folic acid, Vitamin B 12, and a form of methionine known as SAMe (S-adenosylmethionine). All of these compounds function as “methyl donors.” They carry and donate methyl molecules to facilitate necessary chemical processes. The donation of methyl groups by betaine is very important to proper liver function, cellular replication, and detoxification reactions. Betaine also plays a role in the manufacture of camitine. In certain embodiments of Applicant's composition and method, Betaine is provided at between about 31.2 milligrams and about 250 milligrams per administration, i.e. upon ingestion of one dosage of Applicant's composition. In certain embodiments, Applicant's composition includes about 125 milligrams of Betaine.

The amino acid citrulline, compound III, is required to detoxify the liver from ammonia, which is a waste product of the body from oxidation. Citrulline promotes energy and assists with the immune system. This unusual amino acid is formed in the urea cycle by the addition of carbon dioxide and ammonia to ornithine. It is then combined with aspartic acid to form arginosuccinic acid, which later is metabolized into the amino acid arginine.

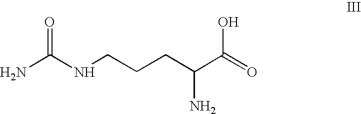

In certain embodiments, Applicant's composition includes Citrulline. Using Applicant's composition and method, Citrulline is provided at between about 3 milligrams and about 25 milligrams per administration, i.e. upon ingestion of one dosage of Applicant's composition. In certain embodiments, Applicant's composition includes about 12 milligrams of Citrulline.

Coenzyme Q₁₀ (also known as CoQ₁₀, Q₁₀, vitamin Q₁₀, ubiquinone, or ubidecarenone) is a compound that is made naturally in the body. Coenzyme Q₁₀ is used by cells to produce energy. It is also used by the body as an antioxidant. An antioxidant is a substance that protects cells from chemicals called free radicals. Using Applicant's composition and method, Coenzyme Q₁₀ is provided at between about 6 milligrams and about 50 milligrams per administration, i.e. upon ingestion of one dosage of Applicant's composition. In certain embodiment, Applicant's composition includes about 24 milligrams of Coenzyme Q₁₀.

Lipoic acid, compound IV, is a widely occurring coenzyme found in most prokaryotic and eukaryotic microorganisms, as well as many plant and animal tissues. Its principle role is in energy metabolism where it is covalently attached, through the carboxylic acid, to Lysine residues forming an essential lipoamide in the E2 subunits of the pyruvate dehydrogenase complex (PDH) and the a-ketoglutarate dehydrogenase complex (KGDH).

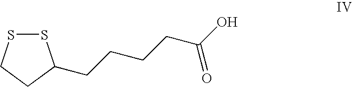

These large multienzyme complexes contain three units: a specialty dehydrogenase (E1p and E1o), a dihydrolipoamide acetyltransferase (E2p and E2o), and a lipoamide dehydrogenase (E3). In the E2p and E2o subunits, the lipoyllysyl moiety forms a “swinging arm” approximately 14 Åin size which aides in the transfer of acyl groups between the various enzymes within the multienzyme complex.

Using Applicant's composition and method, Lipoic acid is provided at between about 24 milligrams and about 200 milligrams per administration, i.e. upon ingestion of one dosage of Applicant's composition. In certain embodiments, Applicant's composition includes about 96 milligrams of Lipoic acid.

Thiamine is a coenzyme for the decarboxylation of pyruvate and the oxidation of alpha keto-glutamic acid. Lipoic acid which is formed in the liver is also required for the reactions. Using Applicant's composition and method, Thiamine is provided at between about 72 milligrams and about 500 milligrams per administration, i.e. upon ingestion of one dosage of Applicant's composition. In certain embodiments, Applicant's composition includes about 288 milligrams of Thiamine.

Further using Applicant's composition and method, Lysine is provided at between about 3 milligrams and about 20 milligrams per administration, i.e. upon ingestion of one dosage of Applicant's composition. In certain embodiments, Applicant's composition includes about 12 milligrams of Lysine.

Creatine, methyl guanidine-acetic acid, shown below as compound V, is formed from three amino acids—Arginine, Glycine and Methionine. Creatine is formed naturally in the liver.

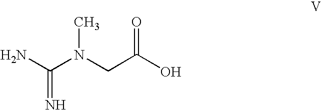

When muscles metabolize ATP for energy, a chemical process takes place wherein the ATP is broken down into two simpler chemicals ADP (adenosine di-phosphate) and inorganic phosphate. This process of ATP turning into ADP releases the energy which gives your muscles the ability to contract. The ADP produced, however, cannot be used to create more energy for the muscles.

Creatine bonds with phosphorus and is converted into Creatine Phosphate (“CP”). CP is able to react with the ADP and turn “useless” ADP back into the “super useful” energy source—ATP. As those skilled in the art will appreciate, additional ATP means more fuel for muscle tissues.

In certain embodiments, Applicant's composition includes Creatine. In certain embodiments, Applicant's composition includes a salt of Creatine, such as Creatine pyruvate. Using Applicants's composition and method, Creatine and/or a Creatine salt is provided at between about 0.5 gram and about 6 grams per administration, i.e. upon ingestion of one dosage of Applicants'composition.

In certain embodiments, Applicant's composition further includes a plurality of digestive enzymes, including Pepsin, Amylase, Lipase, and Protease. Enzymes are catalysts. As those skilled in the art will appreciate, catalysts reduce the activation energy for a process, and hence, increase the reaction rate of that process. As those skilled in the art will appreciate, Amylase, Lipase, and Protease, comprise pancreatic enzymes. As those skilled in the art will further appreciate, proteolytic enzymes, such as Pepsin and Protease, assist protein digestion; amylolytic enzymes, such as Amylase assist in carbohydrate digestion; and lipolytic enzymes, such as Lipase, assist in lipid digestion.

Pepsin and Protease comprise enzymes that assist in the digestion proteins. In the stomach, Pepsin breaks apart proteins at specific amino acid sites. Applicant's composition includes one or more proteolytic enzymes, such as Pepsin and Protease, to facilitate the rapid digestion of proteins, and thereby, increase cellular metabolism. In certain embodiments, using Applicant's composition and method, Pepsin is provided at between about 1.2 milligrams and about 8 milligrams per administration, i.e. upon ingestion of one dosage of Applicant's composition. In certain embodiments, Applicant's composition includes about 4.8 milligrams of Pepsin.

In certain embodiments, using Applicant's composition and method, Protease is provided at between about 6,900 NF units and about 55,000 NF units per administration, i.e. upon ingestion of one dosage of Applicant's composition. In certain embodiments, Applicant's composition includes about 27,600 NF units of Protease.

Applicant's composition includes one or more amylolytic enzymes, such as Amylase. Amylase comprises a key enzyme in digestion of complex carbohydrates, cleaving the—1,4 linkage between glucose residues In the small intestine, Amylase breaks starch into 2-glucose units called maltose. Amylase is made in the pancreas. In certain embodiments, Applicant's composition includes Amylase to facilitate the rapid digestion of starchy materials, and thereby, increase cellular metabolism. In certain embodiments, of Applicant's composition and method, Amylase is provided at between about 7,200 NF units and about 60,000 NF units per administration, i.e. upon ingestion of one dosage of Applicant's composition. In certain embodiments, Applicant's composition includes about 28,800 NF units of Amylase.

Applicant's composition includes one or more lipolytic enzymes, such as Lipase. Lipase combines with a polypeptide, colipase that helps reduce the surface tension at the oil-water interface and helps assist the hydrolysis process. Lipase can then cleave the fatty acids from the glycerol molecule. In certain embodiments, using Applicant's composition and method, Lipase is provided at between about 1,200 NF units and about 10,000 NF units per administration, i.e. upon ingestion of one dosage of Applicant's composition. In certain embodiments, Applicant's composition includes about 4,800 NF units of Lipase.

In certain embodiments, Applicant's composition further includes one or more herbal extracts. By herbal extract, Applicant means a plant with a soft stem containing little wood, where that plant, or one or more portions of that plant, or one or more compounds extracted from that plant, are used for medicinal purposes. For example, in certain embodiments Applicant's composition includes Ginseng. Siberian Ginseng (Eleutherococcus Scenticosus) has long been thought to assist the body in adapting to both internal and external stress, including increasing resistance to environmental stress. Among other things, Siberian Ginseng reportedly enhances physical endurance. Using Applicant's composition and method, Ginseng is provided at between about 60 milligrams and about 0.5 grams per administration, i.e. upon ingestion of one dosage of Applicant's composition. In certain embodiments, Applicant's composition includes about 240 milligrams of Ginseng.

Ligustrum has been found to exhibit several pharmacological properties. These include anti-inflammatory, antimutagenic, and diuretic properties. Ligustrum has been found to help regulate and enhance both liver and kidney function. Using Applicant's composition and method, Ligustrum is provided at between about 60 milligrams and about 0.5 grams per administration, i.e. upon ingestion of one dosage of Applicant's composition. In certain embodiments, Applicant's composition includes about 240 milligrams of Ligustrum.

In certain embodiments, Applicant's composition further includes a third herbal component comprising extracts from the root of Codonopszs pdosula Nannf., and other species of the same genus, family Campanulaceae. This herb is known to be helpful for increasing physical, i.e. athletic, performance. Using Applicant's composition and method, Codonopsis Pilosulae is provided at between about 0.12 gram and about 1 gram per administration, i.e. upon ingestion of one dosage of Applicant's composition. In certain embodiments, Applicant's composition includes about 480 milligrams of Codonopsis Pilosulae.

In certain embodiments, Applicant's composition further includes a fourth herb having the pharmaceutical name Rhizoma Atractylodis macrocephalae, sometimes known by its botanical name Atractylodes macrocephala Koidz or by its common name White atractylodes rhizome. In certain embodiments of Applicant's composition and method, Atractylodis macrocephalae is provided at between about 0.06 gram and about 0.5 gram per administration, i.e. upon ingestion of one dosage of Applicant's composition. In certain embodiments, Applicant's composition includes about 240 milligrams of Atractylodis macrocephalae.

In certain embodiments, Applicant's composition further includes one or more minerals. By “minerals,” Applicant means a compound comprising one or more Transition Metal elements. In certain embodiments, Applicant's composition includes one or more Group VI element/salt and/or one or more Group VIII element/salt. For example, in certain embodiments Applicant's composition includes Chromium and/or one or more Chromium salts. In certain embodiments, Applicant's composition includes Chromium picolinate. Chromium picolinate reportedly stabilizes the body's blood sugar by metabolizing excess fats, carbohydrates and proteins. Such enhanced fat/protein metabolism effectively “spares” glycogen resources, thereby giving enhanced physical endurance and strength. In certain embodiments, using Applicant's composition and method, Chromium is provided at between about 90 micrograms and about 500 micrograms per administration, i.e. upon ingestion of one dosage of Applicant's composition. In certain embodiments, Applicant's composition includes about 360 micrograms of Chromium.

In certain embodiments, Applicant's composition further includes Iron. In certain embodiments, Applicant's composition includes elemental Iron. In certain embodiments, Applicant's composition includes one or more Iron salts. In certain embodiments, Applicant's composition includes one or more Fe⁺⁺ salts. In certain embodiments, Applicant's composition includes one or more Fe⁺⁺⁺ salts. In certain embodiments, Applicant's composition includes Iron citrate. In certain embodiments, using Applicant's composition and method, Iron citrate is provided at between about 3 milligrams and about 25 milligrams per administration, i.e. upon ingestion of one dosage of Applicant's composition. In certain embodiments, Applicant's composition includes about 12 milligrams of Iron citrate.

In certain embodiments, Applicant's composition includes one or more electrolytes. By electrolytes, Applicant means a compound comprising one or more alkaline element, i.e. Group I element, and/or one or more alkaline earth element, i.e. Group II element. In certain embodiments, such one or more Group I element-containing compounds comprise one or more monovalent metal salts. In certain embodiments, such one or more Group II element-containing compounds comprise one or more divalent metal salts. Counterions for such monovalent/divalent metal salts are selected from the group consisting of one or more monovalent anions, including halide, acetate, and the like, one or more divalent anions, including sulfate, and the like, one or more trivalent anions, such a phosphate, and the like, and combinations thereof.

Table I summarizes formulations A, B, C, D, E, F, G, H, and I, of Applicant's composition. As those skilled in the art will appreciate, an effective dosing of Applicant's composition is a function of body weight, fitness level, and environmental factors.

TABLE I

| Component | A | B | C | D | E | F | G | H | I |
|---|---|---|---|---|---|---|---|---|---|
| Methylcobalamine, | 0.6 | 1.2 | 1.8 | 2.4 | 5 | 2.4 | 2.4 | 2.4 | 2.4 |
| mg | | | | | | | | | |
| Chromium, μg | 90 | 180 | 270 | 360 | 500 | 360 | 360 | 360 | 360 |
| Pantathine, mg | 30 | 60 | 90 | 120 | 250 | 120 | 120 | 120 | 120 |
| Niacinamide, mg | 90 | 180 | 270 | 360 | 500 | 360 | 360 | 360 | 360 |
| Biotin, μg | 480 | 960 | 1500 | 1920 | 3000 | 1920 | 1920 | 1920 | 1920 |
| Iron Citrate, mg | 3 | 6 | 9 | 12 | 25 | 12 | 12 | 12 | 12 |
| L-Carnitine, mg | 79.5 | 159 | 240 | 318 | 500 | 318 | 318 | 318 | 318 |
| L-Citrulline, mg | 3 | 6 | 9 | 12 | 25 | 12 | 12 | 12 | 12 |
| α-Lipoic Acid, mg | 24 | 48 | 72 | 96 | 200 | 96 | 96 | 96 | 96 |
| Lysine, mg | 3 | 6 | 9 | 12 | 20 | 12 | 12 | 12 | 12 |
| Coenzyme Q₁₀,mg | 6 | 12 | 18 | 24 | 50 | 24 | 24 | 24 | 24 |
| Thiamine, mg | 72 | 144 | 216 | 288 | 500 | 288 | 288 | 288 | 288 |
| Betaine HCL, mg | 31.2 | 63 | 94 | 125 | 250 | 125 | 125 | | |
| Pepsin, mg | 1.2 | 2.4 | 3.6 | 4.8 | 8 | 4.8 | 4.8 | | |
| Protease, NF units | 6900 | 13800 | 20700 | 27600 | 55000 | 27600 | 27600 | | |
| Lipase, NF units | 1200 | 2400 | 3600 | 4800 | 10000 | 4800 | 4800 | | |
| Amylase, NF units | 7200 | 14400 | 21600 | 28,800 | 60000 | 28800 | 28800 | | |
| Creatine, gm | 0.5 | 1 | 3 | 5 | 6 | | | | |
| Licorice, mg | 60 | 120 | 180 | 240 | 500 | | | | |
| Ligustrum, gm | 0.060 0.12 | 0.18 | 0.24 | 0.5 | | | | | |
| Ginseng, gm | 0.060 0.12 | 0.18 | 0.24 | 0.5 | 0.24 | 0.24 | 0.24 | | |
| Codonopsis Pilosulae, | 0.12 | 0.24 | 0.36 | 0.48 | 1.0 | 0.48 | | | |
| gm | | | | | | | | | |
| Atractylodis | 0.06 | 0.12 | 0.18 | 0.24 | 0.50 | 0.24 | | | |
| Macrocephala, gm | | | | | | | | | |
| Ca⁺⁺/K⁺/Na⁺Mg⁺⁺, | 0.06 | 0.12 | 0.18 | 0.24 | 0.5 | 0.24 | 0.24 | 0.24 | |
| gm | | | | | | | | | |

In certain embodiments, Applicant's composition further includes 7-Keto-Dehydroepiandrosterone. 7-Keto-Dehydroepiandrosterone is a natural metabolite of the human hormone Dehydroepiandrosterone (“DHEA”). DHEA is the most abundant naturally occurring hormone in the body. It is produced by the adrenal gland. It has been reported that DHEA, among other things, increases energy levels. These results are attributed to raised levels of thyroid hormone T3, which increases basic metabolic rate. Using Applicant's composition and method, 7-Keto-Dehydroepiandrosterone is provided at between about 25 milligrams and about 100 milligrams per administration, i.e. upon ingestion of one dosage of Applicant's composition. In certain embodiments, Applicant's composition includes about 50 milligrams of 7-Keto-Dehydroepiandrosterone.

In certain embodiments, Applicant's composition further includes Ephedra. Ephedra is a naturally-occurring substance derived from the Chinese herb ma huang. Ephedra contains two alkaloids, ephedrine and pseudoephedrine. Ephedrine, the main constituent, is a bronchodilator and stimulates the sympathetic nervous system. Pseudoephedrine has a stimulating effect on the heart and blood pressure. Using Applicant's composition and method, Ephedra is provided at between about 3 grams and about 6 grams per administration, i.e. upon ingestion of one dosage of Applicant's composition. In certain embodiments, Applicant's composition includes about 6 grams of Ephedra.

Table II summarizes formulations AA, AB, AC, AD, AE, AF, AG, AH, and Al, of Applicant's composition. As those skilled in the art will appreciate, an effective dosing of Applicant's composition is a function of body weight, fitness level, and environmental factors. The complete Formulation AA comprises the components recited in Table I under the column A in addition to the components recited in Table II under the column AA. Similarly, the complete Formulations AB through Al comprise the components recited in Table I under the columns B through I, respectively, in addition to the components recited in Table II under the columns AB through Al, respectively.

TABLE II

| Component/ | AA | AB | AC | AD | AE | AF | AG | AH | AI |
|---|---|---|---|---|---|---|---|---|---|
| 7-Keto | 25 | 30 | 40 | 50 | 100 | 50 | 50 | 50 | 50 |
| dehydroepi- | | | | | | | | | |
| androsterone, mg | | | | | | | | | |
| Ephedra gm | 3 | 4 | 5 | 6 | 10 | 6 | 6 | 6 | 6 |

In certain embodiments, Applicant's composition comprises a concentrate comprising one of the formulations recited in Table I, and optionally Table II, without water. In other embodiments, Applicant's composition includes water. In certain embodiments, the components recited above are mixed, in any order, with one another using conventional techniques, such as dry blending, compounding, extrusion, and the like. Subsequently, water can be added to that mixture. Alternatively, the components discussed above can be added sequentially to water.

In certain embodiments, the volume of water used is adjusted with respect to the amount of electrolytes and non-electrolytes present to form an aqueous mixture that is isotonic with normal body fluids. In certain embodiments, Applicant's composition is isotonic with normal saline. In certain embodiments, Applicant's composition is isotonic with human blood. As those skilled in the art will appreciate, an isotonic mixture has a concentration of electrolytes and/or non-electrolytes which will exert equivalent osmotic pressure as that solution to which it is compared.

In certain embodiments, Applicant's composition includes no carbohydrates. In certain embodiments, Applicant's composition includes one or more carbohydrates. In such carbohydrate-containing embodiments, the electrolyte levels and carbohydrate levels are adjusted to maximize the gastric emptying time. During moderate exercise, gastric emptying occurs at a rate similar to that during rest; however, more intense exercise appears to inhibit gastric emptying. Evidence indicates beverages containing less than 10% carbohydrate have gastric emptying rates the most similar to water. Several other factors have been shown to impact gastric emptying. Isotonic drinks appear to empty quickly throughout exercise, whereas gastric emptying rate of hypertonic drinks has been shown to decrease over time.

In certain embodiments, Applicant's composition includes Methylcobalamine, Chromium, Pantathine, Niacinamide, Biotin, Iron, Camitin, Citrulline, α-Lipoic Acid, Lysine, Coenzyme Q₁₀, and Thiamine. In certain embodiments, Applicant's composition includes Methylcobalamine, Chromium, Pantathine, Niacinamide, Biotin, Iron, Carnitin, Citrulline, α-Lipoic Acid, Lysine, Coenzyme Q₁₀, Thiamine, and Ginseng. In certain embodiments, Applicant's composition includes Methylcobalamine, Chromium, Pantathine, Niacinamide, Biotin, Iron, Camitin, Citrulline, α-Lipoic Acid, Lysine, Coenzyme Q₁₀, Thiamine, Ginseng, and Betaine. In certain embodiments, Applicant's composition includes Methylcobalamine, Chromium, Pantathine, Niacinamide, Biotin, Iron, Carnitin, Citrulline, α-Lipoic Acid, Lysine, Coenzyme Q₁₀, Thiamine, Ginseng, Betaine, and Pepsin. In certain embodiments, Applicant's composition includes Methylcobalamine, Chromium, Pantathine, Niacinamide, Biotin, Iron, Carnitin, Citrulline, α-Lipoic Acid, Lysine, Coenzyme Q₁₀, Thiamine, Ginseng, Betaine, Pepsin, Protease, Lipase, and Amylase.

In certain embodiments, Applicant's composition includes Methylcobalamine, Chromium, Pantathine, Niacinamide, Biotin, Iron, Carnitin, Citrulline, α-Lipoic Acid, Lysine, Coenzyme Q₁₀, Thiamine, Ginseng, Betaine, Pepsin, Protease, Lipase, Amylase, and Codonopsis Pilosulate. In certain embodiments, Applicant's composition includes Methylcobalamine, Chromium, Pantathine, Niacinamide, Biotin, Iron, Camitin, Citrulline, α-Lipoic Acid, Lysine, Coenzyme Q₁₀, Thiamine, Ginseng, Betaine, Pepsin, Protease, Lipase, Amylase, Codonopsis Pilosulate, and Atractylodis Macrocephala. In certain embodiments, Applicant's composition includes Methylcobalamine, Chromium, Pantathine, Niacinamide, Biotin, Iron, Carnitin, Citrulline, α-Lipoic Acid, Lysine, Coenzyme Q₁₀, Thiamine, Ginseng, Betaine, Pepsin, Protease, Lipase, Amylase, Codonopsis Pilosulate, Atractylodis Macrocephala, and Licorice. In certain embodiments, Applicant's composition includes Methylcobalamine, Chromium, Pantathine, Niacinamide, Biotin, Iron, Camitin, Citrulline, α-Lipoic Acid, Lysine, Coenzyme Q₁₀, Thiamine, Ginseng, Betaine, Pepsin, Protease, Lipase, Amylase, Codonopsis Pilosulate, Atractylodis Macrocephala, Licorice, and Ligustrum.

In certain embodiments, each of the formulations described immediately above may include one or more electrolytes selected from the group consisting of one or more Ca⁺⁺ containing compounds, one or more K⁺ containing compounds, one or more Na⁺ containing compounds, one or more Mg⁺⁺ containing compounds, and mixtures thereof. In certain embodiments, each of the formulations described immediately above may include 7-Keto-Dehydroepiandrosterone Ephedra, and one or more electrolytes selected from the group consisting of one or more Ca⁺⁺ containing compounds, one or more K⁺ containing compounds, one or more Na⁺ containing compounds, one or more Mg⁺⁺ containing compounds, and mixtures thereof.

While the preferred embodiments of the present invention have been illustrated in detail, it should be apparent that modifications and adaptations to those embodiments may occur to one skilled in the art without departing from the scope of the present invention as set forth in the following claims.

I claim:

1. An ingestible composition for enhancing athletic performance, comprising:
Methylcobalamine;
a plurality of B vitamins;
Camitin;
Citrulline;
Thiamine;
Lipoic Acid;
Lysine;
Thiamine;
one or more Group VI elements; and
one or more Group VIII elements.

2. The composition of claim 1, further comprising:
one or more proteolytic enzymes;
one or more amylolytic enzymes;
one or more lipolytic enzymes;
one or more coenzymes; and
one or more herbal extracts.

3. The composition of claim 2, further comprising water, wherein said composition is isotonic with normal saline.

4. The composition of claim 1, wherein said composition includes no carbohydrates.

5. The composition of claim 1, further comprising one or more carbohydrates.

6. The composition of claim 5, wherein said one or more carbohydrates are present up to about 10 weight percent.

7. The composition of claim 1, further comprising:
7-Keto-dehydroepiandrosterone;
Ephedrine; and
Pseudoephedrine.

8. The composition of claim 7, further comprising water.

9. The composition of claim 8, wherein said composition is isotonic with normal saline.

10. The composition of claim 9, wherein said composition includes no carbohydrates.

11. The composition of claim 9, further comprising one or more carbohydrates.

12. The composition of claim 1 1, wherein said one or more carbohydrates are present up to about 10 weight percent.

13. An ingestible composition for enhancing athletic performance, comprising:
Methylcobalamine;
Chromium;
Pantathine;
Niacinamide;
Biotin;
Iron;
Carnitin;
Citrulline;
α-Lipoic Acid;
Lysine;
Coenzyme Q₁₀;
Thiamine;

14. The composition of claim 13, wherein said composition does not include one or more carbohydrates.

15. The composition of claim 13, further comprising:
Betaine;
Pepsin;
one or more enzymes selected from the group consisting of Protease, Lipase, Amylase, and mixtures thereof;
Ligustrum;
Ginseng; and
one or more electrolytes selected from the group consisting of one or more Ca⁺⁺ containing compounds, one or more K⁺ containing compounds, one or more Na⁺ containing compounds, one or more Mg⁺⁺ containing compounds, and mixtures thereof.

16. The composition of claim 15, further comprising creatine.

17. The composition of claim 13, comprising:
about 2.4 milligrams of Methylcobalamine;
about 360 micrograms of Chromium;
about 120 milligrams of Pantathine;
about 360 milligrams of Niacinamide;
about 1920 micrograms of Biotin;
about 12 milligrams of Iron citrate;
about 318 milligram of Camitin;
about 12 milligrams of Citruiline;
about 96 milligrams of α-Lipoic Acid;
about 12 milligrams of Lysine;
about 24 milligrams of Coenzyme Q₁₀; and
about 288 milligrams of Thiamine.

18. The composition of claim 17, further comprising:
about 125 milligrams of Betaine;
about 4.8 milligrams of Pepsin;
about 0.24 grams of Ligustrum; and
about 0.24 milligrams of Ginseng.

19. The composition of claim 13, further comprising:
Codonopsis Pilosulae; and
Atractylodis Macrocephala.

20. The composition of claim 19, wherein:
said Codonopsis Pilosulae is present at about 0.48 grams; and
said Atractylodis Macrocephala is present at about 0.24 grams.

21. The composition of claim 19, further comprising water.

22. The composition of claim 21, wherein said composition is isotonic with normal saline.

23. The composition of claim 13, further comprising:
7-Keto dehydroepiandrosterone; and
Ephedra.

24. The composition of claim 23, wherein said composition does not include one or more carbohydrates.

25. The composition of claim 24, farther comprising water.

26. The composition of claim 25, wherein said composition is isotonic with normal saline.
